(19) United States
(12) Patent Application Publication
(10) Pub. No.: US 2015/0237222 A1
(43) Pub. Date: Aug. 20, 2015

(54) IMAGING MODALITY AND METHOD FOR OPERATING AN IMAGING MODALITY

(71) Applicant: Siemens Aktiengesellschaft, Muenchen (DE)

(72) Inventors: Sultan Haider, Erlangen (DE); Stefan Popescu, Erlangen (DE)

(73) Assignee: SIEMENS AKTIENGESELLSCHAFT, Muenchen (DE)

(21) Appl. No.: 14/624,772

(22) Filed: Feb. 18, 2015

(30) Foreign Application Priority Data
Feb. 18, 2014 (DE) 102014202893.0

(51) Int. Cl. H04N 1/00; A61B 19/00; G06F 3/01

(52) U.S. Cl. CPC H04N 1/00082; G06F 3/011; H04N 1/00005; H04N 1/00029; H04N 1/00061; H04N 1/0035; A61B 19/5225; A61B 19/56; A61B 19/44; A61B 2019/5236; A61B 2019/524; A61B 2019/5276; A61B 2560/0493

(57) ABSTRACT

In an imaging modality and a method for operating an imaging modality with at least one control unit, a mobile control device is used for the at least partial control of the imaging modality.

### BACKGROUND OF THE INVENTION

1. Field of the Invention

The invention relates to a method for operating an imaging modality with at least one control unit, and an imaging modality operated according to such a method.

2. Description of the Prior Art

Imaging modalities such as magnetic resonance systems or X-ray devices are usually situated in an examination room to which a separate, but adjacent, monitoring room is connected. Usually, a patient is received outside the examination room by the operator of the imaging modality, prepared for the examination, and positioned in or on the imaging modality. The operator then leaves the examination room and performs an examination via a local control device. The control device communicates with one or more control units of the imaging modality for performing the examination via cables.

In the case of magnetic resonance systems, usually a control device in the form of a desktop computer with a LCD display is used as a display device. This is accommodated in a monitoring room spaced apart from the examination room with the magnetic resonance system, because the leakage (stray) fields of the magnetic resonance system do not allow the desktop computer to be placed in the vicinity of magnetic resonance system. The desktop computer monitors the operation of the gradient control unit, the radio-frequency coil control unit and further control units of the magnetic resonance system via control signals.

A problem here is that the monitoring of the patient is performed purely visually. In addition, either the patient has to be left alone during the performance of the examination or at least one further person has to assist the operator.

### SUMMARY OF THE INVENTION

An object of the present invention is to provide a method with which the operation of an imaging modality is simplified.

The basis of the invention is considered to be that a mobile control device is used for the operation of the imaging modality instead of a stationary control device, because this increases the radius of action of the operator and in this way simplifies the operation.

THE imaging modality can be an X-ray device, a computed tomography device, a nuclear medicine device such as PET or SPECT, or an ultrasound device. Preferably, the imaging modality IS a magnetic resonance system. In the case of magnetic resonance systems, the presence of operators during ongoing examinations is not problematic, and unlike the case with ultrasound, there is no need for a transducer. Therefore, the freedom of mobility gained is greatest with magnetic resonance systems.

A control unit for the magnetic resonance system can be a gradient control unit or a radio-frequency coil control unit. It can also be a control unit connected upstream of the two aforementioned control units, or other hardware control units.

A tablet computer can be used as the mobile control device, this tablet computer communicating wirelessly with the at least one control unit of the imaging modality. A tablet computer is a portable computer with a touch screen instead of a permanently connected keyboard. Instead of using the keyboard, a tablet computer is controlled using the touch screen and by gestures. It is possible in one embodiment to connect a keyboard, preferably wirelessly, to the tablet computer in order, for example, to write lengthier texts or programming codes. This, however, is not vitally necessary.

Instead of a tablet computer, it is also possible to use other mobile control devices such as smartphones.

A particular problem is that of authentication. Although it is possible for a single source to define an interface for the control units of the imaging modality, which is only known to a few people and is protected as proprietary, this is not practicable for the manufacturers of medical equipment with device sales in the tens of thousands. Therefore, in an embodiment of the invention, access data that permits communication with the at least one control unit is stored on the mobile control device. Therefore, access or communication with the control unit or the control units of the imaging modality is possible only if the control device knows the correct access data, i.e. if the access data are stored or retrievable there. The access data can be a password, a digital code or even personal data, such as biometric data. In order not to significantly delay the data transmission extremely due to lengthy authentication, it is possible, for example, following a first and more intensive authentication, for a session ID, for example, to be issued with which a registered mobile control device will be repeatedly recognized. This prevents any arbitrary control devices made by a manufacturer being able to communicate with any arbitrary imaging modalities.

To protect the mobile control device as such, it is preferable that user identification data enabling access to the mobile control device or a software product, to be stored in a memory of the mobile control device. This also includes storing the user identification data on a server and only loading them for logging-on to the mobile control device. In this case, the mobile control device can be protected as a whole i.e. so that no use is possible without logging-on. However, alternatively or additionally, it is possible for only the software products, also called apps in the case of tablet computers, that permit communication with a control unit of the imaging modality, to be protected with a password. However, the user identification data can also be biometric information, such as fingerprint information or iris information.

Patient identification data can be stored in a memory of the mobile control device. This data is preferably accessible only after release by user identification data. Thus, an operator must first log onto the mobile control device before the patient identification data can be used. The patient identification data is not to permit a log-on, instead this is a way to avoid mishandling or faulty examination. The patient identification data can be used to ensure that the correct patient is examined or operated on and also that the correct examination or operation is performed. This also has the advantage that the same mobile control device is used to check the identity of the patient as that also used to operate the imaging modality. Then, a patient to be examined is identified with the mobile control device.

It is also possible for the patient identification data to be stored for a short time only, and retrieved from a server. A camera, in particular on the mobile control device, an identification card or a barcode can be used for the identification of the patient. The identification unit is preferably embodied as an app on the mobile control device.

Preferably, at least one item of test information can be retrieved can in dependence on the patient identification data obtained. For example, a checklist can be compiled for each patient modified in dependence on the sex, previous illnesses and/or the planned examination or operation. In the case of magnetic resonance examinations, metallic implants are problematic. X-ray examinations are absolutely contraindicated in the event of a pregnancy, unless the patient's life is at risk. On the other hand, this is completely irrelevant in the case of male patients. Contrast media incompatibilities can occur in any examinations.

The use of the imaging modality during interventional procedures has the same advantages, where they are even more important. For example, biometric information can also be used to verify the identity of an anaesthetized patient once again before the start of the intervention. This enables further minimization of mix-ups.

At least one handling instruction or item of handling information can be emitted as an output, dependent on the patient identification data and/or the test information. This can take place acoustically and/or optically. For example, confirmation or selection of a question can be requested. With reference to the previous example, it is possible to ask whether a female patient is pregnant. For selection, in addition to “yes” and “no”, “emergency” can also provide an answer which interrupts or circumvents the standard question. It is also possible for the examination requested by a doctor to be simply displayed as handling information. When a patient has been identified, it is possible, in dependence on the patient, for “abdomen left induration” or “spin-echo abdomen left T2-weighted” to be displayed as the magnetic resonance examination to be performed. Since the mobile control device is also embodied to control the imaging modality, it can additionally suggest a set of parameters, in the above example “TE=150 ms, TR=1.5 s”. These suggestions can be adapted by the operator with the mobile control device.

In the case of an interventional procedure, the handling information can be the intervention to be performed: “stent implantation with catheter”. However, it is also possible for more detailed information about known previous operations of the patient or the like to be displayed.

Preferably, at least one image of an image recording device for monitoring the examination room can be provided as an output on the display of the mobile control device. During the recording of the image data, an operator can, for various reasons, stay at a distance from the imaging modality. Access of the control device to an imaging device enables the examination room to be monitored independently of the presence of the operator in the monitoring or examination room. In particular, this enables the operator, during the performance of an examination on one imaging modality, to accept another patient for examination on another imaging modality. This is, for example, advantageous with magnetic resonance systems since these often have examination times in range of many minutes in which the operator has only to react if the patient suffers a panic attack or the examination is discontinued due to a faulty ECG signal.

Advantageously, the mobile control device can communicate at least one control instruction to the control unit of the imaging modality on the basis of which at least one operating parameter is set or at least one image data set is acquired. Correspondingly, the mobile control device is able to monitor the imaging modality completely.

The control device can be controlled with a voice command and, dependent on the voice command, the control device emits information or a control instruction. In this way, the operator can also control the imaging modality without using the hands, which is in particular advantageous during interventional procedures.

Preferably, the control device can be used to communicate with at least one expert during the examination or an intervention on the patient. If a question should arise during the examination that the operator is unable to resolve alone, the operator can consult an expert. For example, the operator can be a medical technical assistant (MTA). The MTA can then consult a doctor if any problems occur. If the doctor also does not know, it is possible, for example, to ask the head of radiology, etc. Therefore, an expert is a person who is expected to have knowledge in a specific field that the operator does not have. At the same time, the majority of mobile control devices have their own camera so that it is also possible to send real-time image data in addition to the image data of the imaging modality to the expert.

The above object of the present invention is also achieved by an imaging modality that has at least one control unit assigned to the imaging modality for the performance of the method as described.

The implementation of the aforementioned embodiments of the method in the control apparatus can take place as software or as (permanently wired) hardware.

The control device can be embodied as a tablet computer. As described above, this is a mobile computer without a permanently connected keyboard. Although it is possible to connect a keyboard, in particular wirelessly, the tablet computer can generally be operated by means of a touchscreen.

Alternatively or additionally, the mobile control device can have at least one recording device for recording a voice signal. This can be an external or internal microphone. Alternatively, the recording device can be embodied as a headset. Then, the mobile control device, as well as the imaging modality, can be controlled solely by voice commands. This is particularly advantageous in interventional procedures in which the surgeon requires both hands to perform the intervention. It is also possible for an external microphone to be coupled wirelessly to the mobile control device.

At the same time, a mobile control device can also be used for a plurality of imaging modalities. The fact that a mobile control device is assigned to an imaging modality does not mean that this mobile control device cannot also be used with other imaging modalities. In an extreme case, a mobile control device is used by one single operator working on one or more imaging modalities. This operator can then adjust the mobile control device in accordance with the requirements, for example by storing favorites on certain programs or image recording methods. However, the assignment of a mobile control device to one single imaging modality is preferable.

To present handling instructions or for communication with an expert, the mobile control device can comprise have at least one acoustic speaker. The speakers can be arranged internally or externally. In particular, both internal and external speakers can be provided with the external speakers being attached wirelessly and permanently, for example, in the examination room. If the mobile control device moves in a certain circle about the external speakers, they are used to provide speech as an output, otherwise the internal speakers are used. This embodiment is also possible with the microphones.

The imaging modality can be, as described above, an X-ray device, a computed tomography device, an ultrasound device or a nuclear medicine device. If the imaging modality is a magnetic resonance system, for examinations it is preferably embodied as an enclosed system and for interventional procedures preferably as an open system. An open system enables the surgeon to access the patient without having to reposition the patient during image recording.

Preferably, the mobile control device can have at least one memory for storing access data and/or patient identification data. In another development of the imaging modality, this data can be encrypted.

Preferably, at least one image recording device is assigned to the imaging modality with the mobile control device being embodied for communication with the image recording device. The image recording device can also communicate video sequences. Thus, the mobile control device can be used to monitor the examination room as described.

Additionally, the mobile control device can also itself include an image recording device. This is particularly useful for communication with an expert. However, the image recording device can also be used to identify the patient either by means of facial recognition or by means of a barcode attached to the patient.

The mobile control device can include a biometric detection device, in particular a fingerprint scanner. This can register or identify both users and patients. To this end, the biometric detection device is preferably embodied as an app that uses a camera of the mobile control device for recording the recognition data.

Further advantageous embodiments of the method according to the invention correspond to corresponding embodiments of the imaging modality according to the invention.

### BRIEF DESCRIPTION OF THE DRAWINGS

FIG. 1 shows a magnetic resonance system with a mobile control device.

FIG. 2 shows a tablet computer.

FIG. 3 is a flowchart for the operation of an imaging modality in a first embodiment of the invention.

FIG. 4 shows a patient bed.

FIG. 5 shows an X-ray device.

FIG. 6 is a flowchart for the operation of an imaging modality in a second embodiment of the invention.

FIG. 7 is a flowchart for operating an imaging modality in a third embodiment of the invention.

### DESCRIPTION OF THE PREFERRED EMBODIMENTS

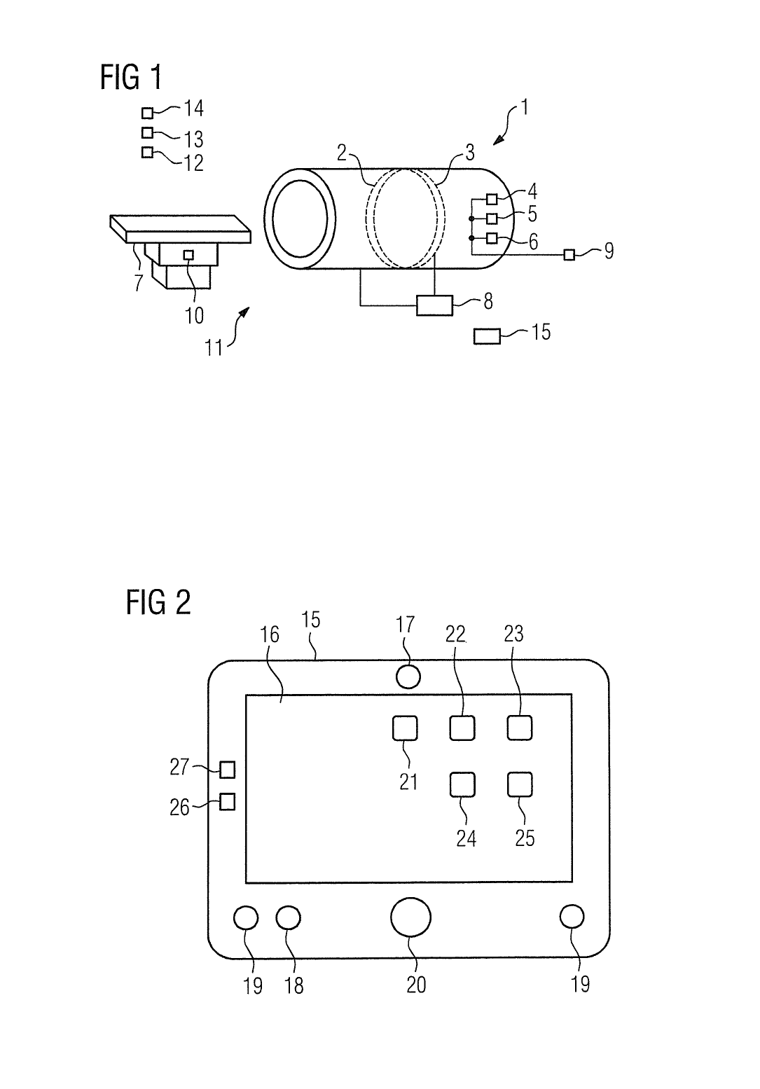

FIG. 1 shows as an embodiment of an imaging modality of a magnetic resonance system 1 with two radio-frequency coils 2 and 3, three gradient coils 4, 5 and 6, a patient bed 7 and three control units 8, 9 and 10. The control unit 8 controls the radio-frequency coils 2 and 3, the control unit 9 controls the gradient coils 4, 5 and 6 and the control unit 10 controls the patient bed 7.

The magnetic resonance system 1 is positioned in the examination room 11. This also contains a camera 12 as an image recording device, a microphone 13 and a speaker 14. The camera 12, the microphone 13 and the speaker 14 can all be connected to other devices, in particular the tablet computer 15, wirelessly by means of Bluetooth.

FIG. 2 shows the tablet computer 15 in detail. This comprises a touch screen 16 as a display and input device, a camera 17, a microphone 18, a speaker 19 and a start button 20. The tablet computer 15 is designed to be MR compatible because it does not have any large metallic components.

Several symbols for apps 21, 22, 23, 24 and 25 are arranged on the touch screen 16. The app 21 can, for example, be an app for patient recognition. When this is activated by tapping the place occupied by the symbol, the tablet computer 15 outputs the handling instruction, either as text or as voice output, to take a photograph of the face of the patient. Obviously, a corresponding memory 26 and microprocessor 27 to store and analyze the camera data are provided. However, the tablet computer 15 can also transmit the camera data for the patient to a server, which carries out the analysis.

When the patient has been identified, further information can be output. If there is to be an examination on the magnetic resonance system 1, it is, for example, possible to ask whether the patient has any metallic implants. In the case of an X-ray system, it is possible to verify that female patients are not pregnant. The patient identification enables patient-specific questions to be created.

The app 22 implements an alternative form of patient identification. Instead of the patient's face, the camera 17 reads out a barcode attached to the patient's wrist. The analysis of a barcode requires less computing power and is therefore advantageous. In addition, there is no need to store biometric patient data, which simplifies the storage of the data with respect to data protection.

The apps 23, 24 and 25 are used to control the magnetic resonance system 1 and to evaluate the data recorded with the magnetic resonance system 1.

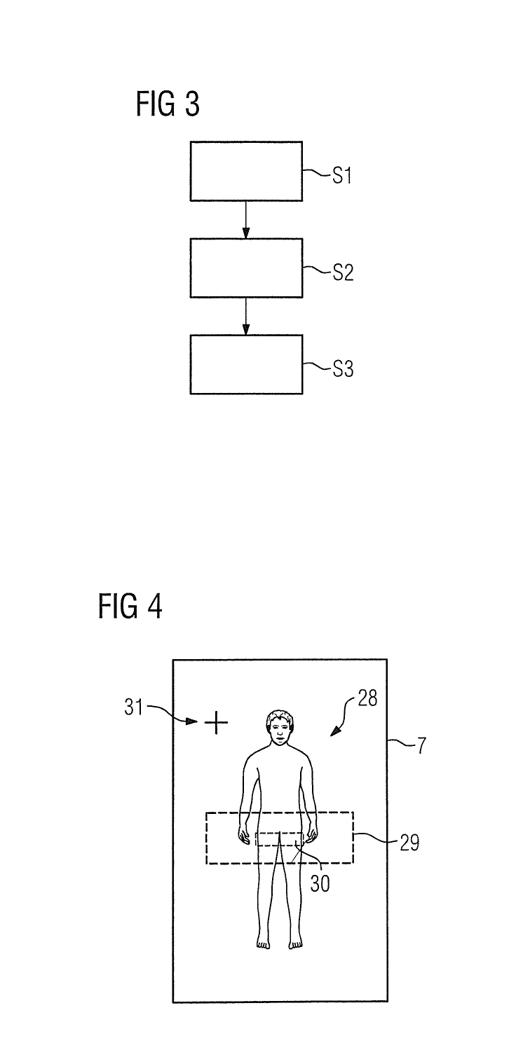

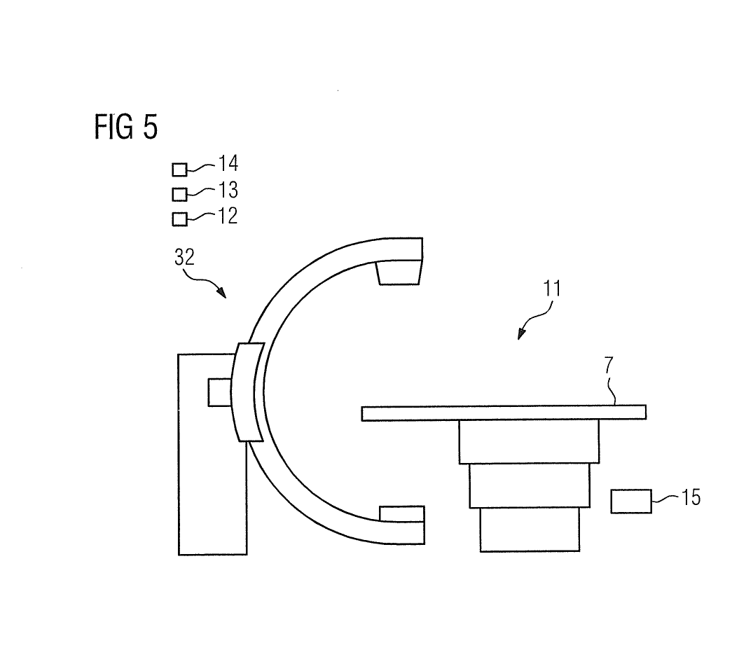

FIG. 3 shows a method for operating a magnetic resonance system 1 in a first embodiment. The explanations are in parallel to those in FIG. 4, which shows a patient 28. In this case, the steps up to the positioning of the patient 28 on the patient bed 7 have been completed. In the first step S1, the operator takes an image of the area to be examined 29 of patient 28. In the image, the slices 30 to be recorded are marked on the tablet computer 15 as step S2. In this case, in a further step S3, all parameters to be set, such as the number of slices, slice thickness, the recording process, for example spin echo or FLASH, etc. can be set. The settings to be derived therefrom such as echo time and repetition time can be checked directly on the tablet computer 15.

To move the patient bed 7 to the correct position, it is possible to make a position marking 31 on the patient bed. To enable this to be used to determine the position, there is a further camera inside the magnetic resonance system 1.

The recording of the camera images can accordingly be performed with the position marking 31. Thus, the tablet computer 15 or any other mobile control device can be used to plan the examination on the patient in advance and not with reference to the image data recorded with the imaging modality.

If a problem should arise during the performance of the examination, the operator can use the tablet computer 15 to consult an expert. During the conversation, the monitoring of the examination room 11 is not lost since the tablet computer 15 is also connected to the camera 12 and/or the camera inside the magnetic resonance system.

FIG. 5 shows an X-ray device 32 for performing an interventional procedure. The tablet computer 15 can also be used to position the patient table 7 as well as to output handling instructions. For example, a surgeon can issue voice commands to the tablet computer 15 in order to move the patient table forward and backward or up or down. Thus, the position of the patient table 7 can also be corrected during an intervention. In particular, it can be provided that a set of voice commands, for example to move the patient table 7, can be blocked with a further voice command in order to avoid injuries to the patient. Otherwise, it may occur that a comment or other remark by a person could be misinterpreted as a command to move the patient table. If, at the time, there is a surgical instrument in the patient's body, the unplanned movement can result in injuries. This feature can obviously be implemented with all imaging modalities and not only with an X-ray device 32.

It is also possible for the control of the X-ray device 32 to use the tablet computer 15 to output handling instructions for the next step of the examination or the next step of the operation.

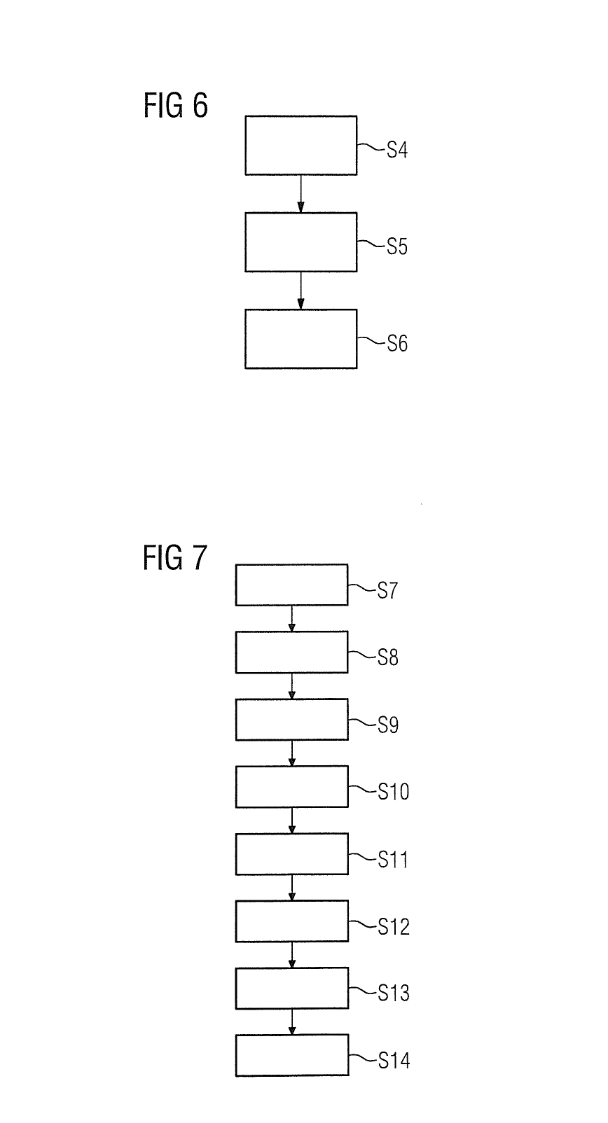

FIG. 6 shows a further flow diagram for the preparation for an examination. In step S4, a barcode attached to the wrist of the patient 28 is recorded with the camera 17 and evaluated with the app 22. In dependence on the patient data, a checklist to be processed is output as step S5. This can in particular also be questions relating to the state of health of the patient which could not be clarified in advance or are dependent on the time of day or even on intolerance of contrast media, in particular magnet resonance contrast media or X-ray contrast media. Only if health-relevant questions are answered satisfactorily or if there is an emergency, will handling instructions for the performance of an examination or operation in step S6 be output on the tablet computer 15.

Alternatively or additionally, information relating to the patient can be output on the tablet computer 15, for example relating to the existence of an additional illness relevant for the purposes of an examination or operation.

In this way, an operator or even a surgeon can be guided through a whole examination or operation in order to minimize handling errors.

A method for operating an imaging modality according to FIG. 7 can have the following features:

Step S7: recording of the patient or the patient's position on the patient table outside the examination or operating room, for example head first or feet first

Step S8: verification of the identity of the patient. As described, this can take place by means of the camera 17 of the tablet computer 15 by means of face recognition or barcode apps.

Step S9: checking questions. Questions relevant for the examination or operation are processed as a checklist on the tablet computer 15. As described, it is possible to investigate whether the patient is pregnant or is intolerant to drugs such as contrast media or narcotics, or has metallic implants etc.

Step S10: emitting handling instructions as an output on the tablet computer 15 for the further preparation of the patient. Since the same tablet computer 15 is used to process the checklist as will be used in the further course of the examination, incorrect instructions will be avoided. For example, the positioning of the patient on the patient bed 7 can be shown on the display 16. It is also possible for the patient 28 with a mobile patient table to be moved from the anteroom into the examination room with it being possible to control the mobile patient table with the tablet computer 15. Alternatively, it is possible to use a wheelchair or a trolley. The tablet computer 15 can be used to check that the wheelchair is MR compatible or X-ray compatible.

Step S11: emitting handling instructions as an output on the tablet computer 15 for the preparation of the imaging modality. In the case of a magnetic resonance system, the coil to be used, its correct positioning and the attachment of further aids, such as an ECG can be displayed.

Step S12: selection of the imaging method. The operator can select the recording method, for example EPI or FLASH. It is also possible to set the recording parameters, such as echo time or repetition time on the tablet computer 15.

Step S13: emitting instructions as an output on how to behave for the patient on the display 16. These instructions can, for example, relate to holding the breath, and, since the recording is planned using the tablet computer 15, also be monitored thereby.

Step S14: emitting handling instructions as an output during the examination. This can relate to the time of administration of a contrast medium or the like.

Here, the tablet computer 15 can be used for each of steps S7 to S14 independently of each other or not; the combinations can be selected as required.

An advantage is that an operator can remain constantly in the vicinity of the patient and does not have to leave the examination room 11 to start recordings. This is in particular useful in the case of people requiring assistance and children.

It is also possible to control a whole examination or an interventional procedure or at least parts thereof with voice input and output.

The patient 28 can also be given a tablet computer. This communicates with the operator's tablet computer 15. The tablet computer of the patient 28 can be used for entertainment and to emit the patient instructions as an output. In this case, the operator does not have to communicate these to the patient 28.

If the tablet computer 15 is used for interventional procedures, the instructions are preferably output by means of voice output via a headset.

Instead of a tablet computer, it is also possible to use other mobile control devices, such as a smartphone. In particular when using a headset and voice input and output, the size of the display 16 is irrelevant.

The tablet computer 15 can also be used for further monitoring tasks. For example, the room temperature of the examination room 11 can be regulated via the tablet computer 15. Both the patient and operators can also be instructed by means of a tablet computer in the event of emergencies, such as the quenching of a magnetic resonance system.

Particularly advantageously, the mobile control device can be connected to information networks such as HIS or RIS. The operator then has access to comparative data and further information.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for operating an imaging modality, said imaging modality comprising at least one control unit, said method comprising:
establishing communication between said control unit and a mobile control device; and
from said mobile control device, remotely controlling at least a portion of operation of said imaging modality by communication between said mobile control device and said control unit.

2. A method as claimed in claim 1 comprising employing a tablet computer as said mobile control device.

3. A method as claimed in claim 1 comprising, in a memory of said mobile control device, storing at least one of user identification data enabling access to the mobile control device, and a software product.

4. A method as claimed in claim 1 comprising, in a memory of said mobile control device, storing access data that allows said communication with said control unit.

5. A method as claimed in claim 1 comprising, at said mobile control device, making an entry that identifies a patient to be examined using said imaging modality and presenting, at said mobile control device, patient identification data dependent on the patient identification entry.

6. A method as claimed in claim 5 comprising, from said mobile control device, retrieving at least one item of test information for said patient, dependent on said patient identification data.

7. A method as claimed in claim 6 comprising providing, at said mobile control device, at least one instruction for implementing examination of said patient by said imaging modality, dependent on at least one of said patient identification data and said test information.

8. A method as claimed in claim 7 comprising providing, at said mobile control device, an image acquired by an image recording device that monitors an examination room in which said imaging modality is situated.

9. A method as claimed in claim 5 comprising providing, at said mobile control device, at least one instruction for conducting said examination of said patient with said imaging modality, dependent on said patient identification data.

10. A method as claimed in claim 1 comprising, from said mobile control device, communicating at least one control instruction to said control unit of said imaging modality that causes at least one operating parameter of said imaging modality to be set, or that causes at least one image data set to be acquired by operation of said imaging modality.

11. A method as claimed in claim 1 comprising controlling at least a portion of operation of said mobile control device dependent on voice commands, and comprising emitting an output signal to said control unit of said imaging modality dependent on said voice command, said output signal comprising information or a control instruction.

12. A method as claimed in claim 1 comprising, from said mobile control device, establishing communication with an expert while conducting an examination of a patient by operation of said imaging modality.

13. An imaging modality comprising:
an image data acquisition unit;
a control unit configured to operate said image data acquisition unit; and
a mobile control device in communication with said control unit, said mobile control device and said control unit being configured to allow at least a portion of operation of said image data acquisition unit to be controlled from said mobile control device.

14. An imaging modality as claimed in claim 13 wherein said mobile control device is a table computer.

15. An imaging modality as claimed in claim 13 wherein said mobile control device comprises at least one image recording device configured to identify a patient to be examined using said image data acquisition unit.

16. An imaging modality as claimed in claim 13 wherein said mobile control device comprises at least one audio recording device that records a voice signal.
